(19) United States
(12) Patent Application Publication
(10) Pub. No.: US 2022/0347338 A1
(43) Pub. Date: Nov. 3, 2022

(54) Air Sanitizing Device

(71) Applicant: Ralph Gregory Greene, Dalton, GA (US)

(72) Inventor: Ralph Gregory Greene, Dalton, GA (US)

(21) Appl. No.: 17/735,071

(22) Filed: May 2, 2022

Related U.S. Application Data
(60) Provisional application No. 63/183,590, filed on May 3, 2021.

(51) Int. Cl. A61L 9/20

(52) U.S. Cl. CPC A61L 9/20; A61L 2209/12; A61L 2209/14; A61L 2209/134; A61L 2209/11

(57) ABSTRACT

An air sanitizing device may include one or more sanitation walls forming a sanitation cavity having a first end and a second end. The sanitation cavity may have an elongated length dimension and a width dimension with the length dimension extending between the first end and the second end. A lateral axis may extend through the sanitation cavity and may bisect the width dimension. At least one elongated Ultraviolet light emitting element may be disposed in the sanitation cavity, preferably parallel to the lateral axis. A plurality of baffles may be coupled to one or more of the sanitation walls and disposed in the sanitation cavity, and one or more baffles of the plurality of baffles may be angled relative to the lateral axis between 25 and 155 degrees. One or more fans may be configured to motivate air around the plurality of baffles and through the sanitation cavity.

### CROSS REFERENCE TO RELATED APPLICATIONS

This application claims priority to and the benefit of the filing date of U.S. Provisional Application No. 63/183,590, filed on May 3, 2021, entitled “Method for increasing UVC dosage in a compact air flow stream”, which is hereby incorporated by reference in its entirety.

### FIELD OF THE INVENTION

This patent specification relates to the field of UVC air sanitizing devices, more specifically a device for treating air with UVC light.

### BACKGROUND

Ultraviolet light has been used extensively for more than 40 years in disinfecting drinking water, waste water, air, pharmaceutical products, and surfaces against a whole suite of human pathogens. While air disinfecting devices that utilize ultraviolet light exist, they suffer from drawbacks and limitations that include: low air flow rates, low sanitation rates, high power consumption, and high noise levels. Additionally, existing devices have low irradiation density and therefore low germicidal action compared to the size of the devices. Irradiation density is expressed in watts per square meter (W/m2). For germicidal action dose is important. The dose is the irradiation density multiplied by the time (t) in seconds and expressed in joules per square meter (J/m2). (1 joule is 1 W.second).

Therefore, a need exists for novel UVC air sanitizing devices which do not suffer from the above-mentioned drawbacks and limitations.

### BRIEF SUMMARY OF THE INVENTION

An air sanitizing device is provided having a plurality of baffles which may be configured to direct medium air traveling across a UV light emitting element perpendicular to the UV light emitting element rather than run down the length of the UV light emitting element. The device may include one or more UV light emitting elements, such that the device may include a second, third, fourth, UV light emitting element to enable the device to have a larger plenum size and/or larger air flow.

In some embodiments, an air sanitizing device may include one or more sanitation walls forming a sanitation cavity, in which the sanitation cavity has a first end and a second end. The sanitation cavity may have an elongated length dimension and a width dimension with the length dimension being greater than the width dimension and the length dimension extending between the first end and the second end. A lateral axis may extend through the sanitation cavity between the first end and the second end, and the lateral axis may bisect the width dimension. At least one elongated light emitting element may be disposed in the sanitation cavity. The light emitting element may emit Ultraviolet light, and the light emitting element may be parallel to the lateral axis. A plurality of baffles may be coupled to one or more of the sanitation walls and disposed in the sanitation cavity, and one or more baffles of the plurality of baffles may be angled relative to the lateral axis between 25 and 155 degrees. One or more fans may be configured to motivate air around the plurality of baffles and through the sanitation cavity.

In further embodiments, the one or more light emitting elements of the device may extend through at least one baffle of the plurality of baffles.

In further embodiments, at least one baffle of the plurality of baffles may extend through the lateral axis.

In still further embodiments, one or more baffles of the plurality of baffles may be angled between 85 and 95 degrees relative to the lateral axis.

In still further embodiments, one or more baffles of the plurality of baffles may be angled between 85 and 5 degrees towards the second end and relative to the lateral axis.

In yet further embodiments, the plurality of baffles may include a second baffle that is adjacent to the first baffle, and the device may include a first dimension describing a distance between a free end of the first baffle and a first sanitation wall opposingly positioned to the free end of the first baffle. The device may also include a second dimension describing a distance between a free end of the second baffle and the first baffle. The first dimension and second dimension may be perpendicular to each other, and the second dimension may be at least 200 percent of the first distance.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a perspective view of an example of an air sanitizing device according to various embodiments described herein.

FIG. 2 illustrates a sectional, through line 2-2 shown in FIG. 1, elevation view of an example of an air sanitizing device according to various embodiments described herein.

FIG. 3 shows an exploded schematic diagram of another example of an air sanitizing device according to various embodiments described herein.

FIG. 4 depicts a partial, sectional view, taken parallel to the lateral axis, of a sanitation cavity of a further example of an air sanitizing device according to various embodiments described herein.

FIG. 5 illustrates a partial, sectional view, taken perpendicular to the lateral axis, of a sanitation cavity of a further example of an air sanitizing device according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

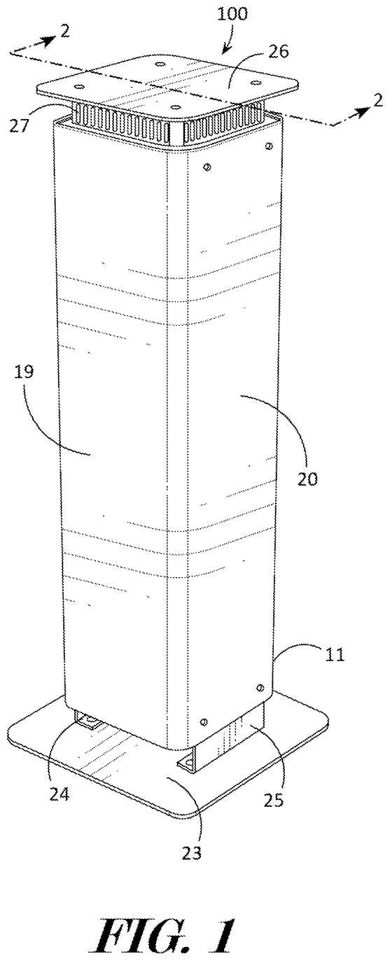

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 10% of the specified number. Additionally, as used in this application, the term “substantially” means that the actual value is within about 10% of the actual desired value, more preferably within about 5% of the actual desired value and even more preferably within about 1% of the actual desired value of any variable, element or limit set forth herein.

A new air sanitizing device is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

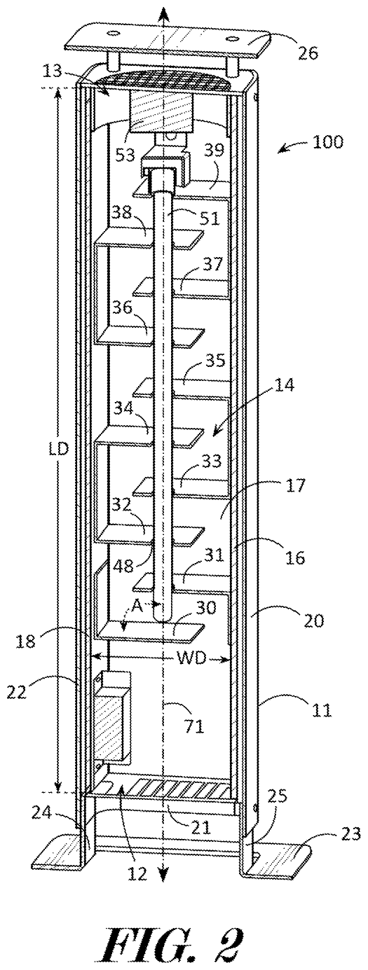

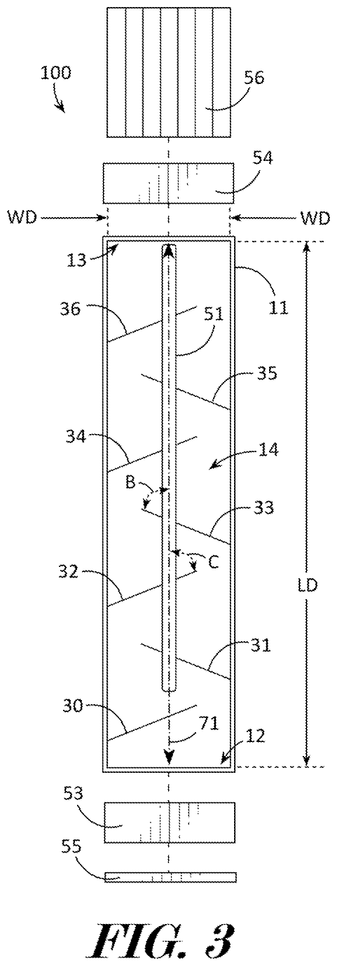

The present invention will now be described by example and through referencing the appended figures representing preferred and alternative embodiments. FIGS. 1-5 illustrate examples of an air sanitizing device (“the device”) 100 according to various embodiments. In some embodiments, the device 100 may comprise one or more sanitation walls 15, 16, 17, 18, forming a sanitation cavity 14, and the sanitation cavity 14 having a first end 12 and a second end 13. The sanitation cavity 14 may have an elongated length dimension (LD) (FIGS. 2 and 3) and a width dimension (WD) (FIGS. 2, 3, and 5), and the length dimension (LD) may be greater than the width dimension (WD) with the length dimension (LD) extending between the first end 12 and the second end 13. A lateral axis 71 (FIGS. 2-5) may extend through the sanitation cavity 14 between the first end 12 and the second end 13, and the lateral axis 71 may bisect the width dimension (WD). An elongated light emitting element 51, 52, may be disposed in the sanitation cavity 14. The light emitting element 51, 52, may emit Ultraviolet light, and the light emitting element 51, 52, may be substantially parallel to the lateral axis 71. A plurality of baffles 30-39, may be coupled to one or more sanitation walls 15, 16, 17, 18, and disposed in the sanitation cavity 14. Preferably, one or more baffles 30-39 of the plurality of baffles 30-39 may be angled relative to the light emitting element between 25 and 155 degrees, and one or more baffles 30-39 of the plurality of baffles 30-39 may extend through the lateral axis 71. One or more fans 53, 54, may be configured to motivate air around the plurality of baffles 30 -39 and through the sanitation cavity 14.

The device 100 may comprise one or more sanitation walls 15, 16, 17, 18, which may be sized and shaped to form a sanitation cavity 14 of any size and shape. In some embodiments, the device 100 may comprise four elongated rectangular shaped sanitation walls 15, 16, 17, 18, which may be coupled together to form a generally elongated rectangular prism shaped sanitation cavity 14. In further embodiments, the device 100 may comprise one or more sanitation walls 15, 16, 17, 18, which may be sized and shaped to form a sanitation cavity 14 having a generally elongated triangular prism shape, elongated cylindrical shape, elongated spiral shape, and even combinations of shapes.

In some embodiments, and as shown in FIG. 5, one or more sanitation walls 15, 16, 17, 18, may comprise one or more projections 28, 29, which may extend towards the one or more light emitting elements 51, 52, to channel or direct the air closer to the one or more light emitting elements 51, 52. Keeping the air close to the one or more light emitting elements 51, 52, greatly increases the UV dosage and efficacy for sanitizing biological contaminants in the air. Projections 28, 29, may be configured in any size and shape and may be positioned anywhere within the sanitation cavity 14.

In some embodiments, the device 100 may comprise a housing 11 which may surround all or portions of the one or more sanitation walls 15, 16, 17, 18, that form a sanitation cavity 14. A housing 11 may include one or more housing walls 19, 20, 21, 22, which may be assembled to form the shape of the housing 11. In some embodiments, a housing 11 may comprise four elongated rectangular housing walls 19, 20, 21, 22, which may be coupled together to form a generally elongated rectangular prism shape housing 11 that may surround the one or more sanitation walls 15, 16, 17, 18, of the sanitation cavity 14. It should be understood that a housing 11 may be configured in any size and shape. For example, a housing 11 may comprise a generally elongated triangular prism shape, elongated cylindrical shape, elongated spiral shape, and even combinations of shapes.

In some embodiments, a housing 11 may comprise a base 23 which may form a stable support for supporting and/or coupling the device 100 on an object, such as a floor, table, wall, ceiling, etc. In some embodiments, a housing 11 may comprise one or more legs 24, 25, which may be couple sanitation walls 15, 16, 17, 18, to the base 23 while separating the sanitation walls 15, 16, 17, 18, a desired distance from the base 23 to facilitate air flow into the first end 12. In some embodiments, the housing 11 may comprise an end cap 26 and/or one or more diffusing apertures 27 which may direct and diffuse air that has passed through the sanitation cavity 14 and is exiting the device 100.

In preferred embodiments, a sanitation cavity 14 may comprise a first end 12 and a second end 13, and the device 100 may be configured to intake air into the sanitation cavity 14 through the first end 12 and exhaust the air from the sanitation cavity 14 through the second end 13. Generally, the first end 12 and second end 13 may comprise and/or may be in communication with one or more apertures of any shape and size in the housing 11 that enable air to be communicated into and out of the sanitation cavity 14.

In preferred embodiments, and as perhaps best shown in FIGS. 2 and 3, the device 100 may comprise a sanitation cavity 14 that may be elongated in shape, such that the sanitation cavity 14 may have an elongated length dimension (LD) (FIGS. 2 and 3) and a width dimension (WD) (FIGS. 2, 3, and 5), and the length dimension (LD) may be greater than the width dimension (WD) with the length dimension (LD) extending between the first end 12 and the second end 13. As an example, the device 100 may comprise a sanitation cavity 14 having a length dimension (LD) that is greater than 200 percent of the width dimension (WD), and more preferably a length dimension (LD) that is greater than 400 percent of the width dimension (WD). The sanitation cavity 14 may also comprise a height dimension (HD) (FIG. 5) with the length dimension (LD) being greater than both the width dimension (WD) and height dimension (HD). It should be understood that the height dimension (HD) and width dimension (WD) may be used interchangeably but are differentiated from the length dimension (LD) by being lesser than the length dimension (LD). The height dimension (HD) and width dimension (WD) need not be equal to each other, but are each less than the length dimension (LD).

The device 100 may comprise a lateral axis 71 that may extend through the sanitation cavity 14 between the first end 12 and the second end 13. A lateral axis 71 may bisect the width dimension (WD) and/or the height dimension (HD). Optionally, a lateral axis 71 may be approximately parallel to the length dimension (LD) and approximately perpendicular to the width dimension (WD) and/or height dimension (HD). In some embodiments, the device 100 may comprise a lateral axis 71 that may be substantially parallel (within plus or minus 5 degrees) to the length dimension (LD). In further embodiments, the device 100 may comprise a lateral axis 71 that may be parallel to the length dimension (LD). It should be understood that the lateral axis 71 need not be linear, but may follow the shape of the width dimension (WD) and/or height dimension (HD). For example, a sanitation cavity 14 that comprises a curved rectangular prism shape will have a curved lateral axis 71 that may approximately bisect the width dimension (WD) and/or the height dimension (HD) along the curved length of the sanitation cavity 14.

The device 100 may comprise one, two, three, four, or more light emitting elements 51, 52, which may be configured to generate Ultraviolet (UV) light that may be emitted into a sanitation cavity 14. The device 100 may include one or more UV light emitting elements 51, 52, such that in some embodiments, the device 100 may include a second, third, fourth, UV light emitting element 51, 52, to enable the device 100 to have a larger sanitation cavity 14, plenum size, and/or larger air flow. UV light may have a spectral range that is between 100 and 400 nanometers (nm). In preferred embodiments, a light emitting element 51, 52, may emit UVC light having a wavelength between 200 to 280 nanometers (nm).

A light emitting element 51, 52, may comprise any device capable of emitting UVC light. In some embodiments, a light emitting element 51, 52, may comprise one or more light emitting diodes (LEDs), organic light-emitting diodes (OLEDs), incandescent light bulbs, fluorescent light bulbs, halogen light bulbs, high-intensity discharge light bulbs, laser light emitters, electroluminescent light sources, neon light sources, or any other type of suitable UVC light source.

A light emitting element 51, 52, may be configured in any shape and size. For example, a light emitting element 51, 52, may comprise a single light bulb or LED that may be coupled to a sanitation wall 15, 16, 17, 18, baffle 30-39, or otherwise disposed in a sanitation cavity 14. As another example, a light emitting element 51, 52, may comprise two or more light bulbs, LED, fluorescent light bulbs, etc., that may be coupled together or which may be located in separate positions in a sanitation cavity 14. In preferred embodiments, a light emitting element 51, 52, may be elongated in shape, such as by having a length dimension that is two or more times, such as by being four times, seven times, ten times, twenty times, fifty times, etc., the size of its width and height dimensions. In further preferred embodiments, the device 100 may comprise an elongated light emitting element 51, 52, disposed in the sanitation cavity 14 in which the light emitting element 51, 52, is substantially parallel (plus or minus five degrees) to the lateral axis 71 by having its elongated dimension substantially parallel to the lateral axis 71.

A light emitting element 51, 52, may be coupled to any element of the device 100 and positioned to emit UV light into the sanitation cavity 14. Preferably, a light emitting element 51, 52, may be positioned or centrally located within the sanitation cavity 14 as shown in FIGS. 2-5. In some embodiments, a light emitting element 51, 52, may be positioned or centrally located within the sanitation cavity 14 by being positioned within 35 to 65 percent of the width dimension (WD) and/or height dimension (HD) from the one or more sanitation walls 15, 16, 17, 18. In preferred embodiments, a light emitting element 51, 52, may be positioned or centrally located within the sanitation cavity 14 by being positioned within twenty five percent, and more preferably within ten percent, of the width dimension (WD) and/or height dimension (HD) from the lateral axis 71.

The device 100 may comprise one or more baffles, such as a first baffle 30, a second baffle 31, a third baffle 32, a fourth baffle 33, a fifth baffle 34, a sixth baffle 35, a seventh baffle 36, an eighth baffle 37, a ninth baffle 38, a tenth baffle 39, etc., that may be disposed or positioned within the sanitation cavity 14 and which may be coupled to one or more sanitation walls 15, 16, 17, 18. A baffle 30, 31, 32, 33, 34, 35, 36, 37, 38, 39, may be coupled a sanitation wall 15, 16, 17, 18, via welding or heat bonding, adhesive, by being integrally formed or molded together, or with any suitable coupling method. A baffle 30-39 may be coupled to one, two, three, or more sanitation walls 15, 16, 17, 18. For example, a device having a sanitation cavity 14 formed with four sanitation walls 15, 16, 17, 18, may have each baffle 30-39 coupled to three sanitation walls 15, 16, 17, 18, with a space between each baffle 30-39 and a sanitation wall 15, 16, 17, 18, that each baffle 30-39 is not coupled to thereby allowing air to pass between each baffle 30-39 and a sanitation wall 15, 16, 17, 18.

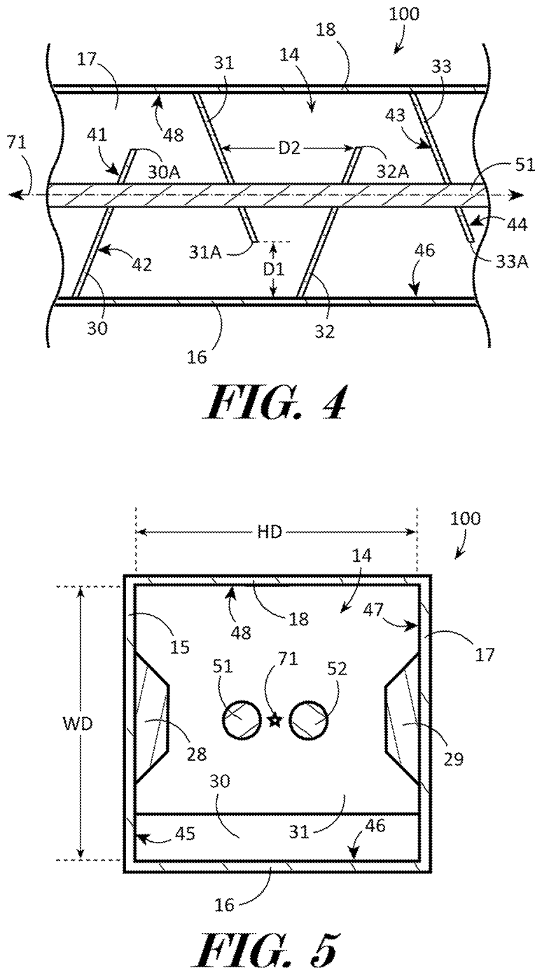

In preferred embodiments, the device 100 may comprise a plurality of baffles 30-39, so as to have at least two baffles 30-39. Generally, baffles 30-39 may prevent air from passing straight through a sanitation cavity 14 by acting as obstacles around which the air must travel to increase the amount of time that the air is in the sanitation cavity 14. In this manner, baffles 30-39 may cause air flowing between the first end 12 and second end 13 to travel in a convoluted path. In preferred embodiments, the device 100 may comprise four sanitation walls 15, 16, 17, 18, and one or more, and more preferably, each baffle 30-39 may be coupled to three sanitation walls 15, 16, 17, 18. Each baffle 30-39 may comprise a free end 30A, 31A, 32A, 33A, (as shown in FIG. 4) that may not be coupled to a sanitation wall 15, 16, 17, 18, thereby forming a space between each baffle 30-39 and at least one sanitation wall 15, 16, 17, 18, to allow air to pass around each baffle 30-39.

Baffles 30-39 may be configured in any size and shape, such as the generally rectangular, planar shaped baffles 30-39 depicted in FIGS. 2-5. In further embodiments, one or more baffles 30-39 may be circular, triangular, flat or planar, or other generally two-dimensional shape, curved, spiral, or other three-dimensional shape, including combinations of shapes.

In preferred embodiments, the device 100 may comprise an elongated light emitting element 51, 52, and the light emitting element 51, 52, may extend through one or more, and more preferably through two or more, baffles 30-39 of the plurality of baffles 30-39 of the device 100. For example, and as shown in FIG. 2, a baffle 32 may comprise a baffle aperture 48 that is sized and shaped to allow a portion of a light emitting element 51, 52, to pass through the baffle 32. Preferably, a light emitting element 51, 52, may not extend through the baffle 30 that is closest to the first end 12. In further preferred embodiments, at least one, and more preferably each, baffle 30-39 of the plurality of baffles 30-39 of the device 100 may extend through the lateral axis 71.

In some embodiments, the device 100 may comprise a plurality of baffles 30-39, and at least one baffle 30-39 of the plurality of baffles 30-39 may be angled relative to the lateral axis 71 between 25 and 155 degrees so that the baffles 30-39 may not be approximately parallel to the lateral axis 71. In further embodiments, the device 100 may comprise a plurality of baffles 30-39, and each baffle 30-39 of the plurality of baffles 30-39 may be angled relative to the lateral axis 71 between 25 and 155 degrees so that the baffles 30-39 may not be approximately parallel to the lateral axis 71. In preferred embodiments, the baffles 30, 39, at each end of the sanitation cavity 14 may be used or configured to block UVC light from exiting the sanitization chamber cavity 14 as UVC light is harmful to the eyes and skin. In further embodiments, the device 100 may comprise any other structure or method of preventing UVC light from exiting the device 100.

Optionally, and as perhaps best shown in FIG. 2, the device 100 may comprise one or more baffles 30-39 which may be approximately perpendicular to the lateral axis 71, such as by being angled between 85 and 95 degrees relative to the lateral axis 71 as shown by angle A in FIG. 2. In some embodiments, the device 100 may comprise a plurality of baffles 30-39, and at least one baffle 30-39 of the plurality of baffles 30-39 may be angled relative to the lateral axis 71 between 25 and 155 degrees so that the at least one baffle 30-39 may not be approximately parallel to the lateral axis 71. In further embodiments, the device 100 may comprise a plurality of baffles 30-39, and each baffle 30-39 of the plurality of baffles 30-39 may be angled relative to the lateral axis 71 between 25 and 155 degrees so that each baffle 30-39 may not be approximately parallel to the lateral axis 71.

Optionally, and as perhaps best shown in FIGS. 3 and 4, the device 100 may comprise one or more baffles 30-39 which may be angled towards the second end 13 as shown by angles B and C in FIG. 3. In some embodiments, the device 100 may comprise a plurality of baffles 30-39, and at least one baffle 30-39 of the plurality of baffles 30-39 may be angled between 85 and 5 degrees towards the second end 13 and relative to the lateral axis 71 so that the at least one baffle 30-39 may not be approximately parallel or approximately perpendicular to the lateral axis 71. In further embodiments, the device 100 may comprise a plurality of baffles 30-39, and each baffle 30-39 of the plurality of baffles 30-39 may be angled between 85 and 5 degrees, more preferably between 75 and 25 degrees, and more preferably between 65 and 45 degrees, towards the second end 13 and relative to the lateral axis 71 so that each baffle 30-39 may not be approximately parallel or approximately perpendicular to the lateral axis 71.

In some embodiments, and as perhaps best shown in FIG. 4, one or more, and optionally each, baffle 30-39 may be angled relative to an adjacent baffle 30-39 while providing a relatively narrow space for air to travel between the baffle 30-39 and a sanitation wall 15, 16, 17, 18, while providing a relatively larger space between the adjacent baffles 30-39 for the air to pass when proximate to a light emitting element 51, 52. For example, the device 100 may comprise a two adjacent baffles 31, 32, and the device 100 may comprise a first dimension (D1) describing a distance between a free end 30A of the first baffle 30 and a first sanitation wall 15 that is opposingly positioned to the free end 30A of the first baffle 30. The device 100 may also comprise a second dimension (D2) describing a distance between a free end 31A of the second baffle 31 and the first baffle 30, and the first dimension (D1) and second dimension (D2) are perpendicular to each other with the second dimension (D2) being at least 200 percent, preferably at least 300 percent, and more preferably at least 400 percent, of the first distance (D1).

Preferably, and as shown in FIGS. 4 and 5, the device 100 may comprise one or more reflective surfaces which may be positioned inside the sanitation cavity 14 and which may increase the amount of UV light that air passing through the sanitation cavity 14 may be exposed to. In some embodiments, and as shown in FIG. 4, one or more baffles 30-39, and more preferably each baffle 30-39, may comprise one or more baffle UVC reflective surfaces 41, 42, 43, 44, that may have greater than 10 percent, more preferably greater than 50 percent, and more preferably greater than 65 percent reflectance of UVC radiation. In some embodiments, and as shown in FIG. 4, one or more sanitation walls 15, 16, 17, 18, and more preferably each sanitation walls 15, 16, 17, 18, may comprise one or more wall UVC reflective surfaces 45, 46, 47, 48, that may have greater than 10 percent, more preferably greater than 50 percent, and more preferably greater than 65 percent reflectance of UVC radiation.

Baffle UVC reflective surfaces 41, 42, 43, 44, and wall UVC reflective surfaces 45, 46, 47, 48, may comprise any material that is capable of reflectance of UVC radiation, such as reflective or polished steel, stainless steel, Tin plate, untreated aluminum, treated aluminum, aluminum sputtered on glass, ‘ALZAK’ treated aluminum, mirrors, mirror coatings, chrome coatings, reflective paints, titanium oxide, magnesium oxide, calcium carbonate, aluminum paint, etc.

In some embodiments, one or more baffles 30-39 and/or sanitation walls 15, 16, 17, 18, may be made from or may comprise a UV transparent or translucent material, such as Teflon or other material.

The device 100 may comprise one or more fans 53, 54, that may be configured to motivate air around the plurality of baffles 30-39 and through the sanitation cavity 14. The one or more fans 53, 54, may be coupled to any element and at any location of the device 100. For example, a device 100 may comprise a fan 53, 54, at or proximate to a first end 12, a second end 13, and/or anywhere between the first end 12 and second end 13 within the sanitation cavity 14.

In some embodiments, the device 100 may comprise one or more filters 55, 56, which may be configured to filter and remove particulate matter in air entering, existing, and or passing through the device 100 depending on location. In some embodiments, the device 100 may comprise a first filter 55 that may function as a pre-filter, so as to filter air entering the device 100, by being coupled to or proximate to the first end 12 so that air passing through the sanitation cavity 14 must first pass through the first filter 55. In some embodiments, the device 100 may comprise a second filter 56 that may function as a post-filter, so as to filter air exiting the device 100, by being coupled to or proximate to the second end 13 so that air exiting the device 100 must first pass through the first filter 55. Optionally, a filter 56 functioning as a post-filter may be configured as an air diffuser and/or sound muffler.

While some exemplary shapes and sizes have been provided for elements of the device 100, it should be understood to one of ordinary skill in the art that the housing 11, sanitation walls 15, 16, 17, 18, baffles 30-39, light emitting element(s) 51, 52 and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 100 may be made from or may comprise durable materials such as aluminum, steel, other metals and metal alloys, wood, hard rubbers, hard plastics, fiber reinforced plastics, carbon fiber, fiberglass, resins, polymers or any other suitable materials including combinations of materials. Additionally, one or more elements may be made from or may comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. An air sanitizing device, the device comprising:
at least one sanitation wall forming a sanitation cavity, the sanitation cavity having a first end and a second end, and the sanitation cavity having an elongated length dimension and a width dimension, wherein the length dimension is greater than the width dimension, and wherein the length dimension extends between the first end and the second end;
a lateral axis extending through the sanitation cavity between the first end and the second end, the lateral axis bisecting the width dimension;
an elongated light emitting element disposed in the sanitation cavity, wherein the light emitting element emits Ultraviolet light, and wherein the light emitting element is parallel to the lateral axis;
a plurality of baffles coupled to the at least one sanitation wall and disposed in the sanitation cavity, wherein a first baffle of the plurality of baffles is angled relative to the lateral axis between 25 and 155 degrees; and
a fan configured to motivate air around the plurality of baffles and through the sanitation cavity.

2. The device of claim 1, wherein the light emitting element emits light having a wavelength between 200 to 280 nanometers.

3. The device of claim 1, wherein the light emitting element extends through the first baffle of the plurality of baffles.

4. The device of claim 1, wherein the first baffle of the plurality of baffles extends through the lateral axis.

5. The device of claim 1, wherein each baffle of the plurality of baffles extends through the lateral axis.

6. The device of claim 1, wherein at least one baffle of the plurality of baffles is angled between 85 and 95 degrees relative to the lateral axis.

7. The device of claim 1, wherein at least one baffle of the plurality of baffles is angled between 85 and 5 degrees towards the second end and relative to the lateral axis.

8. The device of claim 1, wherein the plurality of baffles comprises a second baffle that is adjacent to the first baffle, wherein the device comprises a first dimension describing a distance between a free end of the first baffle and a first sanitation wall opposingly positioned to the free end of the first baffle, wherein the device comprises a second dimension describing a distance between a free end of the second baffle and the first baffle, wherein first dimension and second dimension are perpendicular to each other, and wherein the second dimension is at least 200 percent of the first distance.

9. The device of claim 1, wherein a baffle of the plurality of baffles comprises a baffle UVC reflective surface.

10. The device of claim 1, wherein the at least one sanitation wall comprises a wall UVC reflective surface positioned within the sanitation cavity.

11. The device of claim 1, wherein the light element is centrally located within the sanitation cavity by being positioned within ten percent of the width dimension from the lateral axis.

12. An air sanitizing device, the device comprising:
at least one sanitation wall forming a sanitation cavity, the sanitation cavity having a first end and a second end, and the sanitation cavity having an elongated length dimension and a width dimension, wherein the length dimension is greater than the width dimension, and wherein the length dimension extends between the first end and the second end;
a lateral axis extending through the sanitation cavity between the first end and the second end, the lateral axis bisecting the width dimension;
an elongated light emitting element disposed in the sanitation cavity, wherein the light emitting element emits Ultraviolet light, and wherein the light emitting element is parallel to the lateral axis;
a plurality of baffles coupled to the at least one sanitation wall and disposed in the sanitation cavity, wherein each baffle of the plurality of baffles is angled relative to the lateral axis between 25 and 155 degrees, wherein a first baffle of the plurality of baffles extends through the lateral axis, and wherein the light emitting element extends through at least one baffle of the plurality of baffles; and
a fan configured to motivate air around the plurality of baffles and through the sanitation cavity.

13. The device of claim 12, wherein the light emitting element emits light having a wavelength between 200 to 280 nanometers.

14. The device of claim 12, wherein at least one baffle of the plurality of baffles is angled between 85 and 95 degrees relative to the lateral axis.

15. The device of claim 12, wherein at least one baffle of the plurality of baffles is angled between 85 and 5 degrees towards the second end and relative to the lateral axis.

16. The device of claim 12, wherein each baffle of the plurality of baffles extends through the lateral axis.

17. The device of claim 12, wherein the plurality of baffles comprises a second baffle that is adjacent to the first baffle, wherein the device comprises a first dimension describing a distance between a free end of the first baffle and a first sanitation wall opposingly positioned to the free end of the first baffle, wherein the device comprises a second dimension describing a distance between a free end of the second baffle and the first baffle, wherein first dimension and second dimension are perpendicular to each other, and wherein the second dimension is at least 200 percent of the first distance.

18. The device of claim 12, wherein a baffle of the plurality of baffles comprises a baffle UVC reflective surface.

19. The device of claim 12, wherein the at least one sanitation wall comprises a wall UVC reflective surface positioned within the sanitation cavity.

20. The device of claim 12, wherein the light element is centrally located within the sanitation cavity by being positioned within ten percent of the width dimension from the lateral axis.
